(19) United States
(12) Patent Application Publication
(10) Pub. No.: US 2022/0020452 A1
(43) Pub. Date: Jan. 20, 2022

(54) Systems and Methods for Genetic Comparison and Analysis of Typical and Atypical Tissues Using Machine Learning

(71) Applicants: Richard Kane, Boca Raton, FL (US); Joan M. Greco, Kamuela, HI (US)

(72) Inventors: Richard Kane, Boca Raton, FL (US); Joan M. Greco, Kamuela, HI (US)

(21) Appl. No.: 17/378,937

(22) Filed: Jul. 19, 2021

Related U.S. Application Data
(60) Provisional application No. 63/053,563, filed on Jul. 17, 2020.

(51) Int. Cl. G16B 40/00; C12Q 1/6869; G16B 30/00

(52) U.S. Cl. CPC G16B 40/00; G06N 3/02; G16B 30/00; C12Q 1/6869

(57) ABSTRACT

The present disclosure relates to determining whether a cell is cancerous or suffering from some other condition or not based on real-time comparison to baseline DNA using machine learning tools.

### CROSS-REFERENCE TO RELATED APPLICATIONS

This application claims priority under USC 35 119(e) to U.S. provisional patent application Ser. No. 63/053,563, entitled “DNA Reveal”, filed Jul. 17, 2020, the entirety of which is hereby incorporated by reference.

### FIELD OF THE INVENTION

The present disclosure relates to the detection, identification, and treatment of atypical bodily tissues, including tissues having cancerous cells, using DNA analysis and machine learning systems.

### BACKGROUND OF THE INVENTION

Generally, the detection and identification of cancerous or other atypical cells on body tissues is done by visual inspection followed by biopsy and testing.

Currently, lesions/tumors are incised to determine if there are atypical or cancerous cells present. A piece of the lesion/tumor is then sent to a pathology lab where it undergoes staining and examination. During examination, a pathologist looks at the stained tissue sample under magnification and provides a diagnosis based on their experience observing similar atypical tissues. The examination process itself often takes one to two weeks. If atypical or cancerous cells are identified by the pathologist during examination the patient may then schedule a time for the lesion/tumor to be excised, which could take from days to months. Once the lesion/tumor is excised, doctors will again prep a tissue sample on a slide and visually examine it for signs of atypicality and will continue to remove tissue from around the area in question until the excised cells being examined no longer show the visual characteristics associated with the atypical tissues of the lesion/tumor; however, even then a doctor may not be sure whether or not all of the affected tissues have successfully been removed.

This is a slow and imprecise means for identifying tissues containing atypical cells, identification of the condition resulting in the presentation of atypical cells and determining the margin of any tissues containing atypical cells.

There is a need for a method of rapidly identifying atypical cells in bodily tissues and devising treatments for the condition causing the development or spread of such atypical cells. Additionally, there is the need for a system/method whereby a doctor can determine when a portion of tissue transitions from containing atypical cells to containing only typical cells for that tissue.

### BRIEF SUMMARY OF THE INVENTION

This disclosure covers certain systems and methods for identifying and treating atypical bodily tissues. An exemplary case in which atypical bodily tissues may be treated using the systems and methods disclosed herein is the case of skin cancer.

If a person notices an atypical section of their skin (e.g. a mole, lesion, etc.) a doctor may take a sample of cells from the area in question (a “test sample”) and a sample of cells from an area of the patients skin which has no atypical condition associated with it (a “control sample” or “baseline”), sequence each samples' DNA, and compare the DNA of each of the samples against each other in order to determine their differences. The differences between the DNA code of the control sample and the DNA code of the test sample can be analyzed in order to provide information that may be relevant to treatment of the condition causing the development of atypical tissue.

Doctors may take several samples from an area in question including samples from the margins around the are in question. And may compare their DNA against the control sample. By looking at the differential between the DNA of the test samples and the control sample, a doctor can understand the margins of an area in question more accurately than by other known means. The process of sampling and testing may be iterated until the delta between a test sample and the control sample is within a bounds considered as acceptable, thereby determining the margin of the condition.

The DNA sequences of the control sample and any test samples may be entered into a machine learning program that has been trained on identifying the differences between DNA sequences and their associated conditions, and treatments. The program should be able to then use the comparison between the DNA of the control sample and the DNA of a test sample to quickly associate the mutation of the tissue's DNA sequence with one or more particular skin conditions, and/or characteristics of known skin conditions (e.g. type of cancer, aggressiveness of a tumor, etc.)

This will allow doctors to perform a test capable of determining at a high level of accuracy the type of lesion that the atypical portion of skin comprises, whether or not it requires a biopsy, and in the event of the lesion's excision, when enough tissue has been removed in order to ensure that the margins are clear.

### BRIEF DESCRIPTION OF THE DRAWINGS

The novel features believed characteristic of the disclosed subject matter will be set forth in any claims that are filed later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 shows a flowchart detailing an exemplary embodiment of a method for the DNA sequencing and comparison of atypical tissue cells against a control sample of cells from the same tissue.

FIG. 2 shows a flowchart depicting an exemplary embodiment of a method for comparison of DNA sequences from a test sample and a control sample and providing information related to the differences between the DNA sequences.

FIG. 3 shows a flowchart depicting an exemplary embodiment of a method for determining the margins of a tissue exhibiting one or more atypical characteristics.

FIG. 4 provides a block diagram of an exemplary system for comparing the difference between a test sample DNA sequence and a control sample DNA sequence and analyzing the differences between the two DNA sequences.

### DETAILED DESCRIPTION OF ILLUSTRATIVE EMBODIMENTS

Reference now should be made to the drawings, in which the same reference numbers are used throughout the different figures to designate the same components.

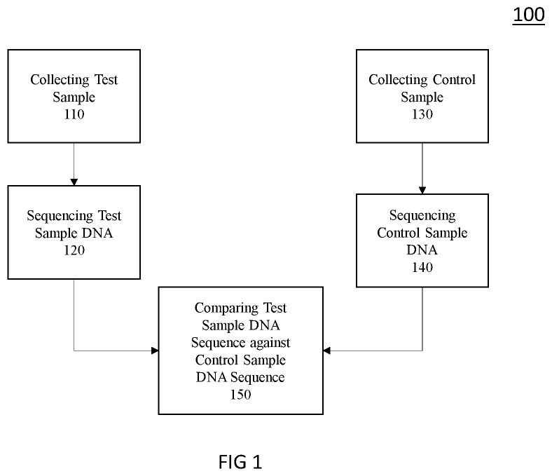

FIG. 1 provides an exemplary method 100 for method for the DNA sequencing and comparison of atypical tissue cells from a tissue test sample against a control sample of cells from the same tissue. Method 100 comprises collecting 110 a test sample comprising cells from a portion of a tissue exhibiting one or more atypical characteristics and collecting 130 a control sample comprising cells from same tissue from which the test sample was collected in step 110 which do not exhibit the one or more atypical conditions present in the area of the tissue from which the test sample was collected. Once collecting 110 the test sample has been completed, method 100 calls for sequencing 120 the DNA of the cells contained in the test sample. Similarly, once collecting 130 the control sample has been completed, method 100 calls for sequencing 140 the DNA of the cells contained in the control sample. Once both sequencing 120 the DNA of the test sample and the sequencing 140 the DNA of the control sample have been completed method 100 calls for comparing 150 the DNA sequence of the test sample against the DNA sequence of the control sample.

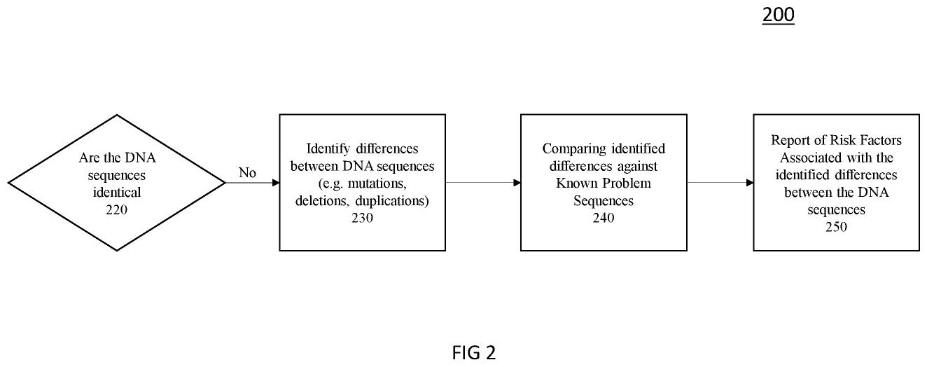

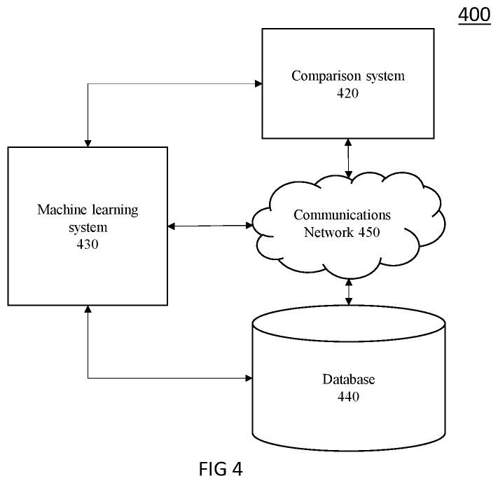

FIG. 2 provides an exemplary method 200 for determining and analyzing the differences between the DNA sequences of a test sample and a control sample. Method 200 may comprise comparing 220 the DNA sequence of a test sample against the DNA sequence of a control sample to determine if the sequences are identical, or if there are differences between the DNA sequences of the two tissue samples. If the two sequences are not identical, method 200 may proceed to identifying 230 the differences between the DNA sequences of the two samples. Once any differences between the DNA sequence of the test sample and the DNA sequence of the control sample have been identified, method 200 may continue to the step of comparing 240 said identified difference against a database containing known problem sequences, such as database 440 of FIG. 4. Once comparing 240 has been completed, method 200 may provide for reporting 250, wherein information related to the known problem sequences having a high level of similarity to one or more of the differences identified between the DNA sequence of the test sample and the DNA sequence of the control sample may be provided from the database.

In embodiments, the differences between the DNA sequence of the test sample when compared against the baseline DNA sequence of the control sample which may be identified by the identifying 230 step of method 200 may comprise the deletion of one or more portions of the DNA sequence of the control sample, the duplication of one or more portions of the DNA sequence of the control sample, the addition of one or more segments of DNA to the DNA sequence of the control sample, and/or other changes in the ordering or positioning of DNA base pairs (collectively, “mutations”).

The database of known problem sequences is of particular diagnostic relevance as specific changes in the DNA of a tissue may cause particular issues or conditions, which may result in significantly different diagnoses and/or treatment requirements. For example, a change in telomerase may be very important to the cancer/non-cancer determination, while variances between the DNA sequences in other areas may be of lesser importance to this determination.

In embodiments, the database used in the step of comparing 240 may further comprise known problematic mutations, as well as characteristics of and treatments for conditions associated with the known problem sequences and/or known problematic mutations.

In embodiments, reporting 250 may comprise the aggregating and display of information such as the level of similarity between the differences between the DNA sequences being tested and any known problem sequences or known problematic mutations contained in the database, along with the conditions, characteristics, and treatment options for known problem sequence or known problematic mutations having a high level of similarity to the differences between the DNA sequence of the test and control samples.

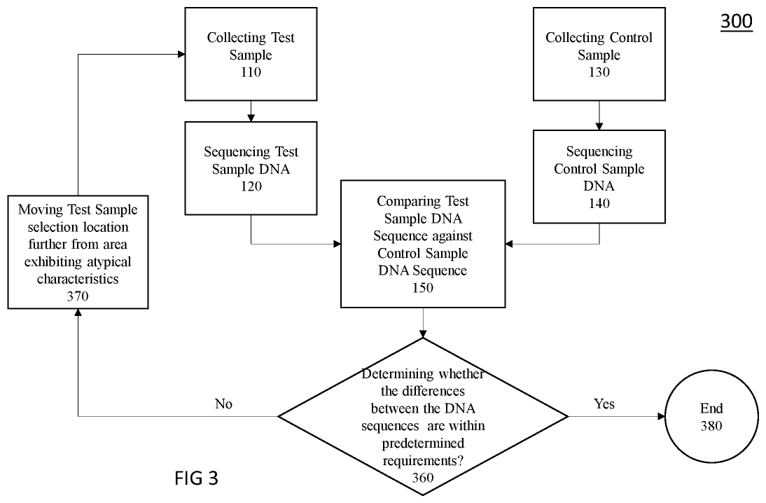

FIG. 3 provides an exemplary method 300 for determining the margins of a tissue exhibiting one or more atypical characteristics. Method 300 comprises the entirety of method 100 discussed hereinabove, including: collecting 110 a test sample comprising cells from an area of a tissue exhibiting one or more atypical characteristics; sequencing 120 the DNA of the test sample collected in step 110; collecting 130 a control sample comprising cells from an are of the tissue that is not exhibiting one or more atypical characteristics; sequencing 140 the DNA of the control sample collected in step 130; and comparing 150 the DNA sequence of the test sample against the DNA sequence of the control sample. Method 300 further comprises the step of determining 360 whether the differences between the DNA sequence of the test sample and the DNA sequence of the control sample are within predetermined requirements. If determining 360 results in a determination that the differences between the DNA sequences of the test control samples are not within the predetermined requirement method 300 may proceed to the step of moving 370 the location from which the test sample is collected to a new test sample collection location located further away, in at least one axis, from the location where the test sample resulting in a negative determination at step 360 was collected. Steps 110, 120, 150, and 360 may then be repeated using a new test sample collected from the new test sample collection location.

Method 300 is structured such that a negative determination at step 360 may begin an iterative loop comprising steps 110, 120, 150, 360, and 370, which once entered may allow for the collection, sequencing, and comparison of multiple test samples from multiple test sample collection locations, each progressively further and further away from the collection location of the initial test sample. This loop formed by steps 110, 120, 150, 360, and 370 of method 300 may be iterated until application of step 360 results in a determination that the DNA sequence of the n^{th} test sample are within the predetermined requirements of the DNA sequence of the control sample, at which point method 300 may end 380.

When used properly and applied repeatedly with the direction of the new test sample selection location established by step 370 changed in order for the updated testing locations to move along various different axes for each application of method 300, the iterative loop structure present in method 300 may allow a doctor to experimentally locate and confirm the margins of an area of an affected tissue.

In embodiments, the predetermined requirements may comprise a threshold percentage difference between the DNA sequences being compared. In alternate embodiments, the predetermined requirements may comprise a requirement that certain segments of DNA code known to be associated with certain condition(s) be completely or substantially identical between the DNA sequences being tested. It should be understood that any suitable set of requirements may be used when applying step 360, and that a person having ordinary skill in the relevant art may be able to configure such requirement in any way that may be required in order to provide suitable differentiation between affected tissue and non-affected tissue.

FIG. 4 provides a block diagram of an exemplary system 300 for comparing and analyzing the differences between the DNA sequence of a test sample and a control sample DNA sequence baseline. The DNA sequence of a test sample and the DNA sequence of a control sample are both loaded into comparison system 420. Comparison system 420 compares the DNA sequence of the test sample against the DNA sequence of the control sample and identifies difference therebetween. The comparison of the DNA sequences performed by comparison system 420 may be consistent with the step of comparing 150 test sample DNA sequence against the control sample DNA sequence from method 100. The differences between the two DNA sequences identified by comparison system 420 may be fed into the machine learning system 430. Machine learning system 430 compares the identified differences between the DNA of the test sample and that of the control sample against database 440, which may contain information related to know genetic mutations in similar tissues, the conditions that are associated with the known genetic mutations, and characteristics of those conditions, which may themselves be associated with a particular genetic mutation. Machine learning system 430 may comprise a neural network that has been trained on database 440 and/or other similar databases in order to be able to rapidly analyze the mutations and provide a user with a listing of conditions, characteristics, and potential treatments that may be associated with said mutations. Machine learning system 430 may then return information from database 440 which it determines to be relevant to one or more of the differences between the DNA sequences of the test sample and the control sample identified by comparison system 420.

Embodiments of system 400 may provide for direct communication between comparison system 420 and machine learning system 430, and between machine learning system 430 and database 440. In alternate embodiments, the component systems of system 400, namely comparison system 420, machine learning system 430, and database 440, may be distributed, in which case said component systems of system 400 may be configured to communicate between one another via a suitable communications network, such as communications network 450.

In embodiments, database 440 may comprise information related to a plurality of tissue conditions. Such information may include DNA sequences associated with tissues experiencing a condition, DNA mutations (i.e. differentials between test sample and control sample DNA sequences) associated with particular skin conditions, information regarding the characteristics of particular skin conditions, and information regarding the mitigation or treatment of particular skin conditions.

In embodiments, the DNA sequences of the control sample and the test sample, along with the differences therebetween, the atypical characteristics of the tissue being tested, and any information related to the tissue's underlying condition, its treatment, and any results thereof may be added to the contents of database 440 in order to provide more data with which machine learning system 430 may be trained and/or against which machine learning system 430 may compare future DNA sequence differentials, in order to improve the accuracy of the analysis performed by machine learning system 430 in the future.

What is claimed is:

1. A method of testing tissues comprising:
1.1. selecting a test portion of a tissue;
1.2. selecting a control portion of the tissue;
1.3. extracting a test sample from the test portion
1.4. extracting a control sample from the control portion;
1.5. sequencing the DNA of the test sample;
1.6. sequencing the DNA of the control sample; and
1.7. comparing the DNA sequence of the test sample to the DNA sequence of the control sample.

2. The method of claim 1, further comprising the steps of:
1.8. determining whether a difference between the DNA sequence of the test sample and the DNA sequence of the control sample are within predetermined requirements.

3. The method of claim 2, further comprising:
1.9. moving the location of the test portion of the tissue responsive to a negative determination at step 1.8; and
1.10. repeating steps 1.1, 1.3, 1.5, 1.7, 1.8, and 1.9 until application of step 1.8 results in a positive determination.

4. The method of claim 1, wherein the comparing step comprises the steps of:
1.7.1. identifying one or more difference between the DNA sequence of the test sample and the NA sequence of the control sample; and
1.7.2. comparing the one or more identified differences against a database containing a plurality of known conditions and one or more known DNA mutations known to be associated with each of the plurality of known conditions.

5. The method of claim 1, wherein the tissue is an animal tissue.

6. The method of claim 1, wherein the test portion of the tissue comprises a characteristic, and wherein the control portion of the tissue does not comprise the characteristic.

7. A method of determining the margin around an area of tissue having atypical DNA characteristics, comprising: the steps described in claim 2.

8. A system for analysis of differences between a test DNA sequence and a control DNA sequence comprising:
a database comprising:
information about a plurality of tissue conditions
an identification system configured to:
receive the test DNA sequence and the control DNA sequence;
compare the test DNA sequence to the control DNA sequence; and
identify differences between the test DNA sequence and the control DNA sequence;
a machine learning system configured to:
receive the differences between the test DNA sequence and the control DNA sequence from the identification system;
compare the differences between the test DNA sequence and the control DNA sequence against the information contained in the database; and
return a portion of the information associated with one or more tissue conditions having genetic characteristics related to the differences between test DNA sequence and the control DNA sequence.

9. The system of claim 8, wherein the machine learning system comprises a neural network.

10. The system of claim 8, wherein the machine learning system comprises an artificial intelligence system.

11. The system of claim 8, wherein one or more of the database, the identification system, and the machine learning system are distributed and are communicably connected via a communications network.
